(19) United States
(12) Patent Application Publication
(10) Pub. No.: US 2009/0306614 A1
(43) Pub. Date: Dec. 10, 2009

(54) TOP LAYER FOR AN ABSORBENT ARTICLE

(75) Inventor: Elisabeth Boissier, Vallda (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 12/306,004

(22) PCT Filed: Jun. 30, 2006
(86) PCT No.: PCT/SE2006/000815

§ 371 Date: Dec. 22, 2008

(51) Int. Cl. A61F 13/15

(52) U.S. Cl. 604/367; 604/378

(57) ABSTRACT

An absorbent product such as a panty liner or a sanitary towel or the like having two lateral zones and situated between them a central zone, the absorbent product having a top layer consisting of a laminate of a first layer of material containing openings and a second layer of material. The first layer of material covers at least the two lateral zones, and in that the second layer of material covers both the two lateral zones and the central zone, the top layer being folded over itself in such a way that an overlap is formed with a Z-shaped cross section, the overlapping parts being joined together via an inner joining zone. A method for the manufacture of such an absorbent product.

### TECHNICAL FIELD

The disclosure relates to an absorbent product such as a panty liner, incontinence pad, sanitary towel or the like comprising a top layer consisting of a laminate of a first layer of material containing openings and a second layer of material, the absorbent product exhibiting two lines extending in the longitudinal direction, which divide the product in a lateral direction in such a way that the product exhibits a first lateral zone, a second lateral zone and positioned between them a central zone. The disclosure also relates to a method for the manufacture of an absorbent product of this kind.

### BACKGROUND ART

In previously disclosed absorbent products, the top layer comprises a layer of material that is liquid-permeable in order to be able to transport liquid from the top layer to the subjacent absorption body. The liquid can be menstrual fluid or urine, and the choice of the material used for the top layer depends on which of these liquids the absorbent product is primarily intended to take up. When choosing the material, consideration must also be given to the required suitability of the top layer having regard for the need for the wearer to experience the material as comfortable against his/her body, and for the wearer to experience a sensation of dryness after the liquid has been excreted. A hydrophobic material is already known to give such a dry sensation. Also previously disclosed is the embodiment of a water-impermeable layer of material with holes in order to make the top layer permeable to air and water, at the same time as the water-impermeable material in other respects prevents the reflux of moisture from the absorption body.

The problems associated with the choice of material for the top layer are particularly great in the lateral zones, because the lateral zones of the absorbent product make contact at least partially with a wearer's groin. The nature of the problem is that the groin moves relative to the lateral zones and can give rise to chafing, and that the lateral zones become crumpled. Movement by the wearer also causes the absorbent product to describe simultaneous movement in the lateral zones, which imposes high demands on the material in order for the material not to rupture. The movement in the lateral zones is usually less than the movement in the central zone, which can give rise to chafing on the wearer if an inappropriate material is used in the lateral zones.

WO 03/015681 depicts an absorbent product comprising a top layer of a film or nonwoven containing a plurality of holes, the holes being larger in the central zone than in the lateral zones. The small holes in the lateral zones are provided to give the material a softer feel compared with a non-perforated material. One problem with holes is that the holes are always open and permit excessive reflux of moisture from the absorption body. Another problem is that the holes permit leakage of material lying loosely under the top layer, for example superabsorbents (SAP), fibres, odour inhibitors, etc. Superabsorbents are small balls or grains of an absorbent material lying loosely in the absorption body, which can be transformed into a gel-like substance by the absorption of water. Any material lying loosely that has found its way out through the holes ends up between the absorbent product and the wearer's skin and can cause discomfort for the wearer, for example in the form of chafing. The above-mentioned problems are especially great in the lateral zones of the absorbent product, because the movement of the lateral zones affects the absorption body in such a way that SAP and liquid tend to be transported out through the holes by the movement in the lateral zone. Making the holes so small that the superabsorbents are not able to leak out through the holes, or so small that the moisture reflux problem is reduced, would reduce the transport of liquid from the top layer to the absorption body, that is to say the admission capacity of the top layer to the subjacent material, to an unacceptably low level. Similar problems exist for all particles that risk leaking out through the holes, for example the above-mentioned particles.

A wish and a need accordingly remain for an absorbent product, in which the above problems are overcome by means of a top layer having improved characteristics in the lateral zones as far as the minimal leakage of loosely lying material is concerned. A further wish is for the top layer, at least in the lateral zones, to present a soft and comfortable surface to the wearer. Additional desirable advantages are for the lateral zones to possess a high degree of air permeability in order to reduce the amount of moisture present in the groin of a wearer. There is also a desire for a simple and inexpensive method for the manufacture of such an absorbent product.

### SUMMARY

The disclosure proposes to solve the above-mentioned problems by means of an absorbent product such as a panty liner, incontinence pad, sanitary towel or the like comprising a top layer consisting of a laminate of a first layer of material containing openings and a second layer of material. The absorbent product exhibits, theoretically, two lines extending in the longitudinal direction, which divide the product in a lateral direction in such a way that the product exhibits a first lateral zone, a second lateral zone and positioned between them a central zone. The first layer of material is advantageously attached to the second layer of material, either fully or partially.

The first layer of material covers the two lateral zones, and in that the second layer of material covers both the two lateral zones and the central zone, the top layer being folded over itself along an inner fold line and an outer fold line, which outer and inner fold lines are present in a boundary zone between one of the lateral zones and the central zone in such a way that an overlap is formed with a Z-shaped cross section, the overlapping parts being joined together adjacent to at least one of the lines extending in the longitudinal direction in an inner joining zone.

The expression openings is used here to denote holes or slits, and embodiments of the disclosure that are described below in conjunction with the use of holes are also eligible for slits.

The expression boundary zone is used here to denote that the theoretical subdivision of the absorbent product into zones. The boundary zone consists in practice of a floating transition between the lateral zones and the central zone, in which boundary zone the Z-shaped, folded part of the top layer is present. The boundary zone can also be defined as that part of the absorbent product that is present between the two fold lines. The overlapping parts are joined together in an inner joining zone present in the boundary zone.

According to a preferred embodiment, the overlapping part of the Z-shaped fold is oriented inwards towards the central zone. The expression overlapping is used here to denote that it lies over a part of the top layer, and that the upper part of the top layer is intended to face towards a wearer during use.

The expression fold line is used here to denote an envisaged line, along which the first layer of material is folded. The fold line thus need not take the form of a variation present in the layer of material, for example a compressed part or the like, although it not precluded from including such a variation.

The expression absorbent product is used here essentially to denote a material or a material composition with the ability to retain liquid for a predetermined period.

The first layer of material is advantageously arranged on the absorbent product in such a way that it faces towards a wearer during use.

The subdivision of the absorbent product into zones and sections is of a theoretical nature without distinct boundaries and is performed for the sole purpose of facilitating understanding of the disclosure. The longitudinally extending lines can thus be straight or curved, depending on the design of the product, in conjunction with which the central zone is not restricted to a square or rectangular geometry, but can exhibit any appropriate geometry, for example circular, oval, triangular, etc. The lateral zones thus have a corresponding design at the interface with the central zone and in a corresponding manner follow the contour of the absorbent product, that is to say they follow the edge part of the absorbent product.

One advantage of embodiments of the disclosure is that loose particles present under the top layer are prevented from finding their way out through the top layer via the holes, at the same time as holes are permitted in the first layer of material, with the result that a textile and comfortable feel for the wearer is imparted to the top layer. Loose particles can be, for example, superabsorbents or particulate odour inhibitors or loose fibre material, or which can otherwise pass through the holes and by so doing find their way out into the surrounding area, which can give rise to irritation for the wearer.

Another advantage of embodiments of the disclosure is that the top layer can be manufactured separately and then joined together with other appropriate parts in an absorbent product, for example a backing layer and/or an absorption body.

According to one embodiment of the disclosure, the top layer comprises a first part containing the first layer of material consisting of a material with holes of a first size and a second part containing the first layer of material comprising a material with holes of a second size. The material in the first part can be the same as in the second part, or it can be a different material. The first part covers at least the two lateral zones, and the second part covers the interjacent central zone. The top layer is folded in such a way that the first part overlaps a section of the second part in such a way that the overlap forms a Z-shaped cross section.

According to another embodiment of the disclosure, the first part of the first layer of material contains holes, and the second part either lacks a first layer of material or comprises a first layer of material but without holes. The top layer is folded in such a way that the first part overlaps a section of the second part in such a way that the overlap forms a Z-shaped cross section.

According to one embodiment of the disclosure, the fold lines consist of two essentially parallel fold lines to either side of the central zone. The two fold lines are present at a distance from one another both in the lateral sense (i.e. the plane of the top layer) and in the vertical sense (i.e. in a direction which coincides with the thickness of the top layer). The fold line that is present closest to the central zone is referred to below as the inner fold line, and the fold line that is present furthest away from the central zone is referred to below as the outer fold line.

The inner joining zone can be positioned adjacent to the inner fold line, in which case the overlap is locked in position with a small pleat, folded over the inner fold line, in the overlapping part that is present and is oriented towards the central zone.

The inner joining zone can be positioned adjacent to the outer fold line, in which case the overlap is locked in position with a large pleat in the overlapping part that is present and is oriented towards the central zone. One advantage of this positioning of the inner joining zone is that the pleat can be filled with a material in order to enable the pleat to function as a barrier, so-called “standing gathers”, which prevents liquid from being transported in a direction away from the central zone to the lateral zones. The material can be an elastic fibre, or an elastic foam, or an absorbent material, or some other suitable material.

The inner joining zone can be positioned between the outer and the inner fold lines, in which case the overlap is locked in position with a pleat in the overlapping part that is present against the central zone having a size in between the extremities indicated above. One advantage of this positioning of the inner joining zone is that the size of the pleat can be determined in accordance with desired parameters.

According to one embodiment, the absorbent product includes a backing layer, to which the top layer is attached via an outer joining zone. The top layer includes an outer edge part on the opposite side of the fold lines, and the top layer is advantageously attached to the backing layer adjacent to this outer edge part. The absorbent product can also include an absorption body between the top layer and the backing layer. The outer joining zone in this case can also include an outer part of the absorption body.

According to one embodiment of the disclosure, the absorbent product comprises a receiving layer, to which the top layer is attached via the inner joining zone. One advantage of this embodiment is that the receiving layer and the top layer can be manufactured as a unit, which can subsequently be applied to any desired core element, for example consisting of an absorption body and/or a backing layer, etc. The top layer can also be attached directly to the backing layer or to the absorption layer via the inner joining zones.

The disclosure also relates to a method for the manufacture of an absorbent product, such as a panty liner or a sanitary towel or the like, comprising a top layer containing two lateral zones and between them a central zone. The method involves the lamination of the top layer, starting with a first layer of material and a second layer of material. The first layer of material is advantageously attached directly to the second layer of material.

Embodiments of the method are characterized in that the first layer of material is laminated on top of the second layer of material in such a way that it covers at least the two lateral zones of the absorbent product, and in that the second layer of material covers the whole of the absorbent product, in conjunction with which the top layer is folded over itself along fold lines that are present in a boundary zone between the lateral zones and the central zone, in such a way that an overlap is formed having a Z-shaped cross section, the overlapping parts being joined together via an inner joining zone.

One advantage of the method is that the top layer can be manufactured separately in the form of a web, the first layer of material and the second layer of material being manufactured individually, after which they are laminated in the desired manner. The method offers the advantage that the position of the perforated part can be controlled by simple means in relation to the non-perforated part. If perforation were to be performed in situ after the top layer had been arranged on the core element of the absorbent product, it would be very difficult to make holes only in the first layer of material.

In conjunction with the manufacture of a separate top layer according to the disclosure, it is easy to use an optionally prefabricated first layer on the basis of desired parameters in respect of the plurality of holes, the width of the first part and the second part, etc., and then to fold the first material along the line which separates the first part from the second part. The method thus provides the manufacturer with the opportunity to resort to the use of a subcontractor with simple specifications for a flat layer to the desired design. It can be stated by way of example that this freedom of choice means that the manufacturer of the absorbent product can select an appropriate width for the first part and the second part respectively in order to permit one fold, two folds, three folds or more folds. A plurality of folds gives a thickness in the first layer of material with the ability to form a barrier with the associated advantages indicated above.

Joining can take place by gluing; or by welding by means of ultrasonic or laser; or by mechanical joining, for example in the form of embossing or compression, etc.; or by some other appropriate method of joining, for example thermal bonding.

The absorption body is appropriately manufactured from a suitable fibre material, in the form of natural or synthetic fibres having absorbent properties, or a mixture of natural fibres and synthetic fibres or other absorbent materials of a previously disclosed kind that are suitable for use in sanitary towels, incontinence pads and panty liners, for example. The absorption body can also contain a predetermined proportion, for example 20-60%, of superabsorbent materials, for example polymer materials in the form of particles, fibres, flakes or similar, which have the ability to absorb and to chemically bind liquid equivalent to several times their own weight while forming an aqueous gel. This provides a very high water-absorbent capacity in the finished product.

It must also be noted that the absorption body can exhibit different forms, for example an essentially elongated and rectangular form, or alternatively some other more irregular form, for example hourglass or triangular form. The absorption body also preferably has rounded edges.

The liquid-permeable first and second layer of material, which together form the top layer, preferably consist of the same material or a combination of the following materials: a fibrous material, for example a soft nonwoven material, although it can alternatively consist of other materials or material laminates. The first layer of material is preferably fully or partially perforated, that is to say holes have been made in the layer, although the second layer of material can alternatively be entirely unperforated, perforated solely in the central zone, or perforated for the full extent of the product. The first layer of material and the second layer of material can appropriately consist of a perforated plastic film, for example a thermoplastic plastic material such as polyethylene or polypropylene, or a mesh-like layer of synthetic or textile material or a perforated nonwoven material. Synthetic fibres such as polyethylene, polypropylene, polyester, nylon or the like, are preferably used as a nonwoven material. Mixtures of different types of fibres can also be used for the aforementioned nonwoven material. The disclosure is not, however, restricted in principle to use only for top layers which consist of nonwoven material, but can also be applied in conjunction with the processing of other materials, for example films made of thermoplastics such as polyethylene or polypropylene.

The disclosure can also be implemented with a top layer which has different types of laminates or combinations of laminates. For example, the top layer can have a number of different laminates which cover parts of the product. In the event that the product has a plurality of laminates, for example divided up into a plurality of longitudinal sections having different laminate sections, these different laminate sections can have different materials and can possess different characteristics. For example, each laminate section can then have different types of perforation, hole positioning, dimensions, hydrophobicity, etc. The different laminate sections can then be joined together by means of ultrasonic welding in a previously disclosed manner that is not described here in detail.

The liquid-permeable top layer is preferably manufactured from a material that exhibits characteristics such as dryness and softness during the time when the absorbent product is being worn, because this top layer is in contact with the wearer's body. It is also desirable for the top layer to have a soft and textile-like surface which remains dry, even in the event of repeated wetting. The top layer can consist of a nonwoven material, for example, with a soft and smooth surface, such as a spunbond material made from polypropylene fibres. A perforated, hydrophobic nonwoven material may be used as the firs layer of material in order to permit the surface that is closest to the wearer's body to be kept dry, in conjunction with which holes are formed in the material that are larger than the distance between the fibres in the material. In this way, liquid can be led down through the holes in the top layer to the subjacent absorption core. Other examples of materials for the top layer are perforated plastic films such as a perforated polyester film. The top layer can be joined together with the subjacent backing layer and the absorption core, for example by means of adhesive, ultrasonic joining or by means of some form of thermal bonding.

The top layer can contain small perforations which surround the holes in the first layer of material with the intention of further increasing the intake of liquid and the acquisition characteristics. It is desirable for the thickness of the top layer to be as great as possible, because a great thickness has a positive effect on the acquisition.

The top layer can also be a three-dimensional laminate of nonwoven and plastic film or a carded, thermally bonded material based 100% on polypropylene. The plastic film can be hydrophilic, pre-perforated (with small holes) and manufactured from a mixture of polyethylene and polypropylene. The nonwoven materials can have a weight per unit area in the range from 12-100 gsm, and in particular in the range from 15-60 gsm.

The nonwoven part of the top layer can also be a spunbond nonwoven material, an air-thru nonwoven material, a spunlace nonwoven (hydroentangled) material, a meltblown nonwoven material, or a combination of these. The raw material can be polypropylene (PP), polyethylene (PE) polyester (PET), polyamide (PA), or a combination of these. If a combination is used, this can be a mixture of fibres from different polymers, although each fibre can also contain different polymers (for example PP/PE bi-component fibres or PP/PE copolymers). Where appropriate, the plastic film can consist of PE or PP, PET, PLA or amyl (or any other thermoplastic polymer), or a mixture or copolymers of the aforementioned polymers.

The perforated first layer of material can also be manufactured from a single layer of material, such as a nonwoven material or a film (as described above).

The holes in the top layer can be oval and slightly elongated in the direction of the machine. The holes can be round/circular or oval in the direction of the machine or in the transverse direction. In addition to the advantages indicated above, the holes impart a textile and comfortable feel to the top layer.

The holes can also be replaced by slits, which by definition differ from the holes in that the slits do not constitute constant openings, but instead are through going incisions in the layer of material. The slits are opened and closed by movement in the material. According to the disclosure, the slits are from 2 mm up to 15 mm in length, and preferably lie in the range from 3-10 mm. The length of the slits and the distance between the slits and the direction of the slits varies depending on the material in the top layer, because the natural tendency of the slits to open depends on the characteristics of the material present in the top layer.

The distance between the slits is in the order of 5-15 mm, although this is dependent on a range of factors, for which reason the distance between the slits can vary depending, among other things, on the material in the top layer and the length of the slits and the direction of the slits. This distance must be sufficiently great between the slits to prevent the top layer from being torn apart when the wearer moves, and sufficiently great to allow the slits to close in the desired manner without the influence of other slits, although at the same time sufficiently small for the ability to breathe and the liquid permeability to remain at an acceptable level. The durability of the top layer is largely governed, however, by the relationship between the surface containing slits and the surface without slits for a given material strength, where the distance between the slits is a subset of the parameters for the durability.

The open surface in the top layer can lie in the range from 2 to 60%, and preferably from 5 to 30%, and in particular preferably from 12 to 16%.

The backing layer is preferably liquid-impermeable (or at least possesses high resistance to penetration by liquid) and is thus so arranged as to prevent any leakage of excreted fluid from the product. The backing layer, on the other hand, may be executed so that it is vapour-permeable. For this purpose, the backing layer may be manufactured from a liquid-impermeable material which consists appropriately of a thin and liquid-proof plastic film. For example, plastic films of polyethylene, polypropylene or polyester can be used for this purpose. Alternatively, a laminate of nonwoven and plastic film or other suitable layers of material can be used as a liquid-proof backing layer. In a previously disclosed manner, the under side of the backing layer can be provided with beads of adhesive or some other previously disclosed attachment means, which can then be utilized for the application of the product to an item of clothing. The product can also be provided with wings, that is to say folding flaps which, in a previously disclosed manner, are arranged along the sides of the product and can be utilized in conjunction with the application of the product.

The product also includes a further layer of material in the form of a receiving layer (also referred to as an acquisition layer, an admission layer and a distribution layer, depending on the function of the material). The receiving layer can be in the form of a wadding material having an appropriately specified thickness and resilience, which in this case is intended to be positioned between the absorbent core and the top layer. The receiving layer possesses essentially the same dimensions as the top layer, with the exception of its thickness, however, which can deviate from the thickness of the top layer. It is also possible to establish that the receiving layer can consist of materials other than wadding material. For example, it may consist of a so-called airlaid material, which is usually based on cellulose fibres.

The receiving layer can appropriately be a porous, elastic, relatively thick layer of material, for example in the form of a fibrous wadding material, a carded fibre wadding, a tow material, or some other kind of bulky and/or resilient fibre material with a high instantaneous liquid intake capacity that is capable of storing liquid temporarily before it is absorbed by the subjacent absorbent core. The receiving layer can also consist of a suitable quantity of thermoplastic fibres in order to permit ultrasonic welding. The receiving layer can also be in the form of a porous foam material. It can also have two or more layers of material. According to a preferred embodiment, the receiving layer can extend towards the lateral edges of the product, that is to say it possesses essentially the same form as the top layer. In this way, advantages can be achieved in respect of liquid distribution, edge sealing, etc.

When manufacturing the absorbent product, the top layer is joined to the backing layer and can also be joined to the receiving layer and/or the absorption body. Joining can take place by gluing; or by welding by means of ultrasonic or laser, or by mechanical joining, for example in the form of embossing or compression, etc.; or by some other appropriate method of joining, for example thermal bonding.

It must be stated, however, that the choice of material and the thickness and density of the layer of material may change in the future in the event of changed manufacturing methods and new material combinations, as a consequence of which the disclosure is not restricted to the materials and material combinations indicated above.

### BRIEF DESCRIPTION OF DRAWINGS

The disclosure is described below in conjunction with preferred illustrative embodiments shown in the accompanying drawings, in which:

FIG. 1 depicts schematically a view from above of an absorbent product in accordance with the disclosure;

FIG. 2 depicts schematically a sectional view along the line A-A in FIG. 1 according to a first embodiment of the disclosure;

FIG. 3 depicts schematically a sectional view along the line A-A in FIG. 1 according to a second embodiment of the disclosure;

FIG. 4 depicts schematically a sectional view along the line A-A in FIG. 1 according to a third embodiment of the disclosure;

FIG. 5 depicts schematically a sectional view along the line A-A in FIG. 1 according to a fourth embodiment of the disclosure.

### DETAILED DESCRIPTION

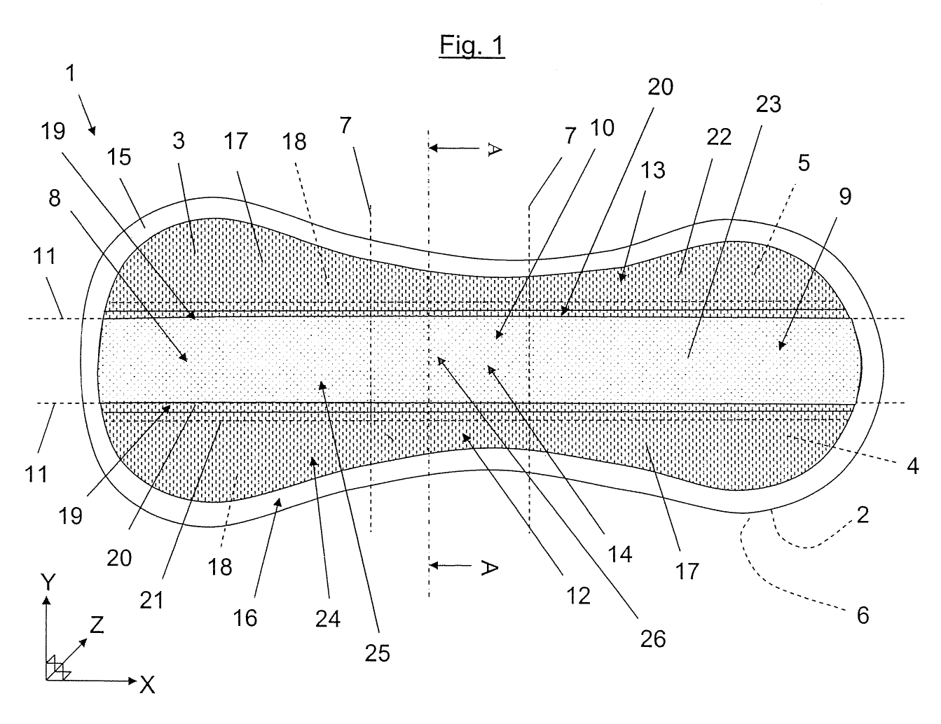

FIG. 1 depicts schematically a view of the top layer of an absorbent product 1 according to the disclosure. The absorbent product 1 here is a sanitary towel, which can be used as undergarment protection in conjunction with menstruation or light incontinence. The absorbent product 1 is positioned in a plane position and exhibits in this plane an extent in the longitudinal direction, the X-axis in the Figure, and in the longitudinal direction, the Y-axis in the Figure, and has a thickness perpendicular to the plane, the Z-axis in the Figure. When wearing the absorbent product 1, the absorbent product 1 is formed around the lower abdomen of a wearer.

The absorbent product 1 comprises a backing layer 2, a top layer 3 and between them an absorption body 4. The absorption body 4 has a first surface 5 and a second surface 6, in conjunction with which the backing layer 2 is arranged over the second surface 6 of the absorption body 4, and the top layer 3 is arranged over the first surface 5 of the absorption body 4. Depicted in FIG. 1 are two laterally extending lines 7, which theoretically divide the product in the longitudinal direction into a rear section 8, a front direction 9 and between them a central section 10. Depicted in FIG. 1 are two lines 11 extending in the longitudinal direction, which divide the product in the lateral direction described by a first lateral zone 12 and a second lateral zone 13 and positioned between them a central zone 14. FIG. 1 shows that the absorbent product 1 is bounded by a peripheral edge part 15. FIG. 1 also shows that the top layer 3 is joined to the backing layer 2 in outer joining zones 16 which coincide with the peripheral edge part 15.

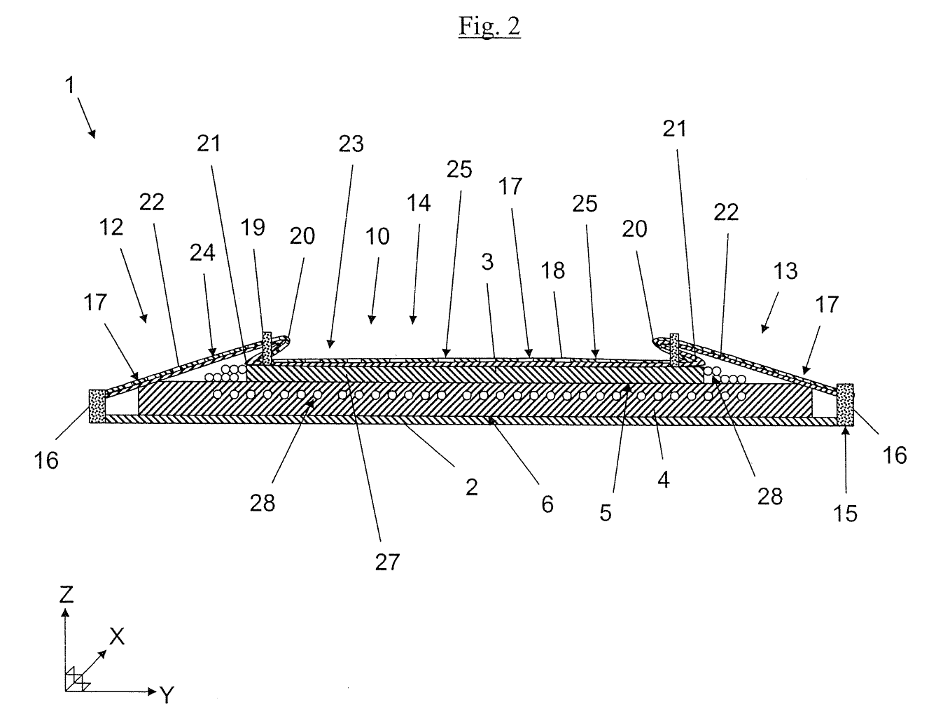

The top layer 3 comprises a first layer of material 17 positioned over a second layer of material 18 in such a way that a laminate is formed by the two layers. The top layer 3 extends essentially over the whole of the absorbent product 1, although the first layer of material 17 can be varied within the scope of the disclosure, and examples of different variations are described below in conjunction with FIGS. 2-5.

A feature common to all the embodiments, however, is that the top layer is folded in a special way, and that the folding is secured by means of inner joining zones 19. The top layer 3 is folded over an inner fold line 20 and an outer fold line 21 in such a way that a Z-shaped cross section is obtained. The top layer contains a first part 22, which in FIG. 1 coincides with the lateral zones 12, 13.

The top layer 3 also includes a second part 23, which in FIG. 1 coincides with the central zone 14. According to FIG. 1, the first layer of material contains a plurality of holes 24 in the first part 22. Depending on the embodiment, the second part 23 can also contain a plurality of holes 25 of the same or different size. In a similar fashion, the first layer of material 17 can contain different materials in the first part 22 and the second part 23 respectively.

The second layer of material 18 lacks holes and is intended to prevent loose particles that are present beneath the top layer 3 from migrating out from the absorbent product 1 via the holes 24 and, in appropriate cases, the holes 25.

The first layer of material 17 contains in the second part material and holes 25 that are particularly adapted to the so-called wet area 26. The wet area 26 is an area in the absorbent product 1 which substantially coincides with that part of the absorbent product 1 that is formed by the sections through the central zone 14 and the central section 9 and can vary depending on the primary area of application of the absorbent product 1, for example light incontinence protection or menstruation protection. When using the absorbent product 1 as incontinence protection, the wet area 26 is that part of the absorbent product 1 which, when it is being used by a wearer, lies facing towards and in close proximity to the wearer's urethra. The wet area 26 thus constitutes the part of the absorbent product 1 that will receive the largest flow of liquid in conjunction with the excretion of liquid waste by the wearer. In the case of menstruation protection, the centre of the wet area 26 can be displaced somewhat in relation to the urethra in such a way that the wet area 26 is positioned against that part of a women's lower abdomen from which the flow of menstruation fluid is discharged.

The disclosure is not restricted to the first layer of material 17 being positioned in the lateral zones 12, 13, but the first layer of material 17 can also be positioned in the rear section 8 and the front section 9.

FIG. 2 depicts schematically a sectional view along the line A-A in FIG. 1 according to a first embodiment of the disclosure. FIG. 2 depicts the first layer of material 17 and the second layer of material 18 as having an identically large extent over the absorbent product. The first layer of material 17 contains, in the lateral zones 12, 13 and thus in the first part 22, a plurality of holes 24 of a first size which give the top layer a textile and comfortable feel for the wearer. The first layer of material 17 contains in the central zone, that is to say the second part 23, a plurality of holes 25 of a different size than in the first part 22. FIG. 2 shows that the first layer of material 17 consists of the same material in the first part 22 and in the second part 23. The first layer of material 17 need not consist of the same material, but can comprise a first material in the first part and a second material in the second part.

FIG. 2 shows that the first layer of material 17 is positioned on the upper side of the absorbent product 1, that is to say on the part of the absorbent product 1 that is intended to face towards a wearer during use. The perforated first part 22 is intended here to give the material in the lateral zones 12, 13 a textile and comfortable feel for the wearer. The second layer of material 18 is arranged between the first layer of material 17 and the subjacent laminated structure consisting of a receiving layer 27, the absorption body 4 and the backing layer 2.

FIG. 2 shows that the absorption body 4 contains superabsorbents 28 (hereinafter referred to as SAP) in the form of small granules. FIG. 2 also shows that the SAP granules 28 have migrated from the absorption body 4 in such a way that they are lying freely under the top layer 3 in the lateral zones 12, 13. The SAP granules migrate in the absorption body depending on the construction and structure of the absorption body. The second layer of material 18 prevents the SAP granules 28 from escaping through the top layer 3 into the lateral zones 12, 13, which means that the top layer 3 can contain holes 24 for imparting the textile feel.

As mentioned above, the absorbent product 1 includes a receiving layer 27 positioned between the top layer 3 and the absorption body 4. The inner joining zones 19 extend in the Z-axis through both the first layer of material 17 and the second layer of material 18 and at least partially through the receiving layer 27. An advantage is achieved in the manufacturing process by attaching the top layer 3 to the receiving layer 27, or to the absorption body 4 when no receiving layer 27 is present, because the receiving layer 27 at the time of manufacture is advanced in a well-defined path, which imparts a fixed position to the top layer 3 relative to the receiving layer 27 in a finished element. A further advantage is that the produced element can be attached simply to the so-called core element, which consists of the other layers present in the absorbent product 1, that is to say the backing layer 2 and the absorption body 4 or, in an appropriate case, the backing layer 2 alone. The receiving layer 27 consists of a material with an open structure which permits the rapid transport of liquid from the top layer 3 to the subjacent absorption body 4. The receiving layer 27 can possess a high or a low distribution capacity, although it will always transport liquid effectively from the top layer 3 to the absorption layer 4. The task of the receiving layer 27 is to ensure that the wearer experiences a feeling of dryness once liquid has been transported away from the top layer 3.

FIG. 2 shows that the inner joining zones 19 bond the top layer 3 and the receiving layer 27 together between the inner fold line 20 and the outer fold line, essentially parallel along the longitudinally extending lines 11. The outer joining zone 16 bonds the first part 21, the second part 22 and the backing layer 2 together. The outer joining zone 16 can also contain the absorption body 4, in which case the edge part 15 possesses a softer feel than when only the top layer 3 and the backing layer 2 form the edge part 15.

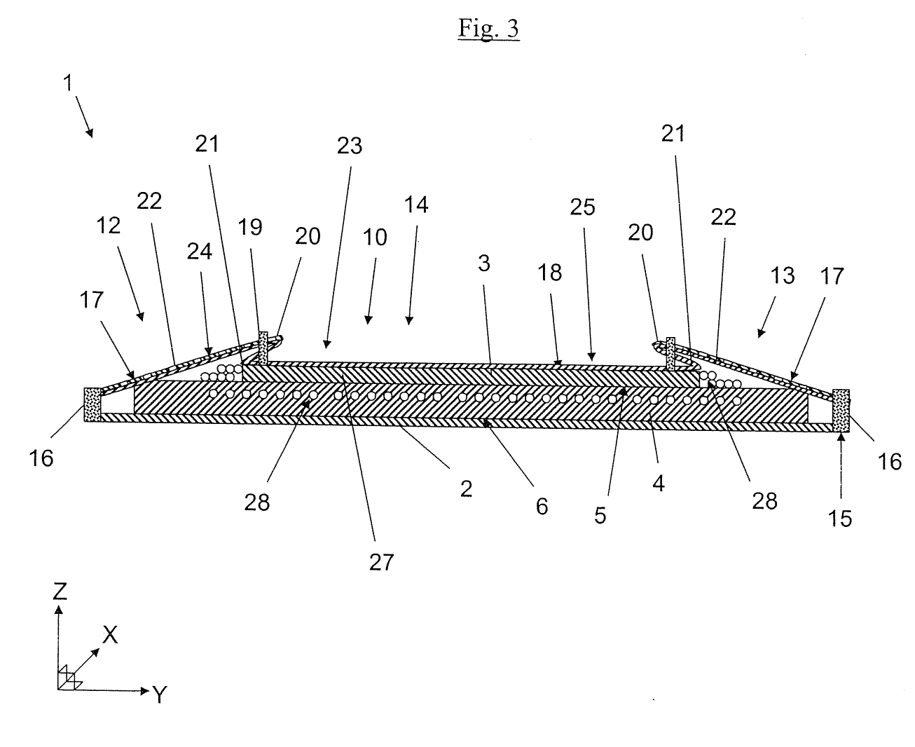

FIG. 3 depicts schematically a sectional view along the line A-A in FIG. 1 according to a second embodiment of the disclosure. FIG. 2 and FIG. 3 are identical apart from the fact that the first layer of material in FIG. 3 lacks the holes 25 that are depicted in FIG. 2.

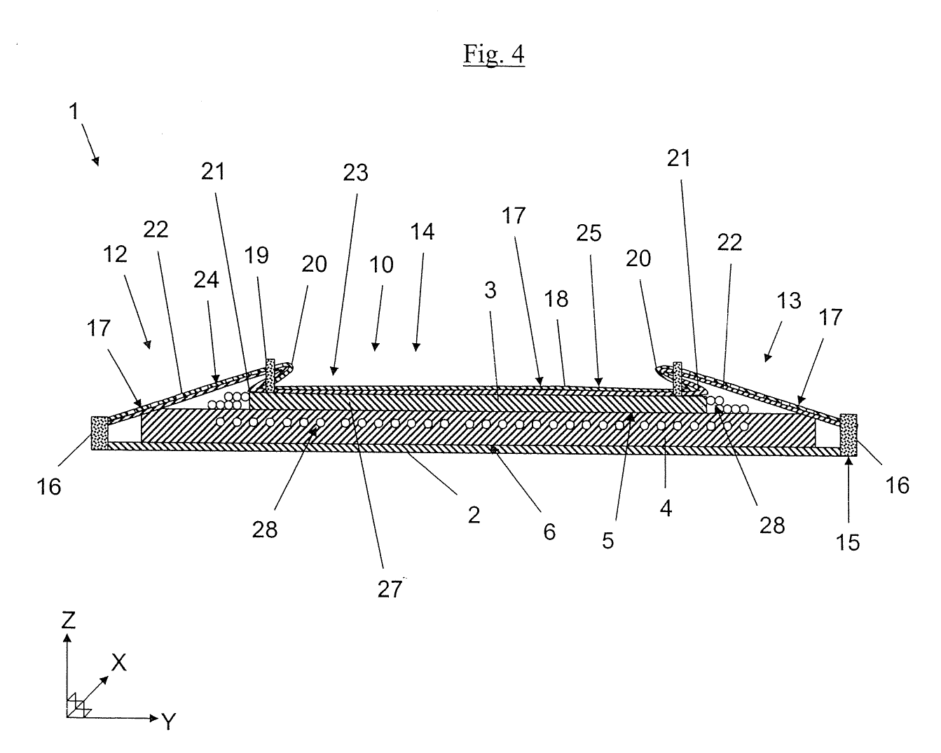

FIG. 4 depicts schematically a sectional view along the line A-A in FIG. 1 according to a third embodiment of the disclosure. FIG. 2 and FIG. 4 are identical apart from the fact that the first layer of material in FIG. 4 is entirely absent in the central zone 14, that is to say in the second part 23.

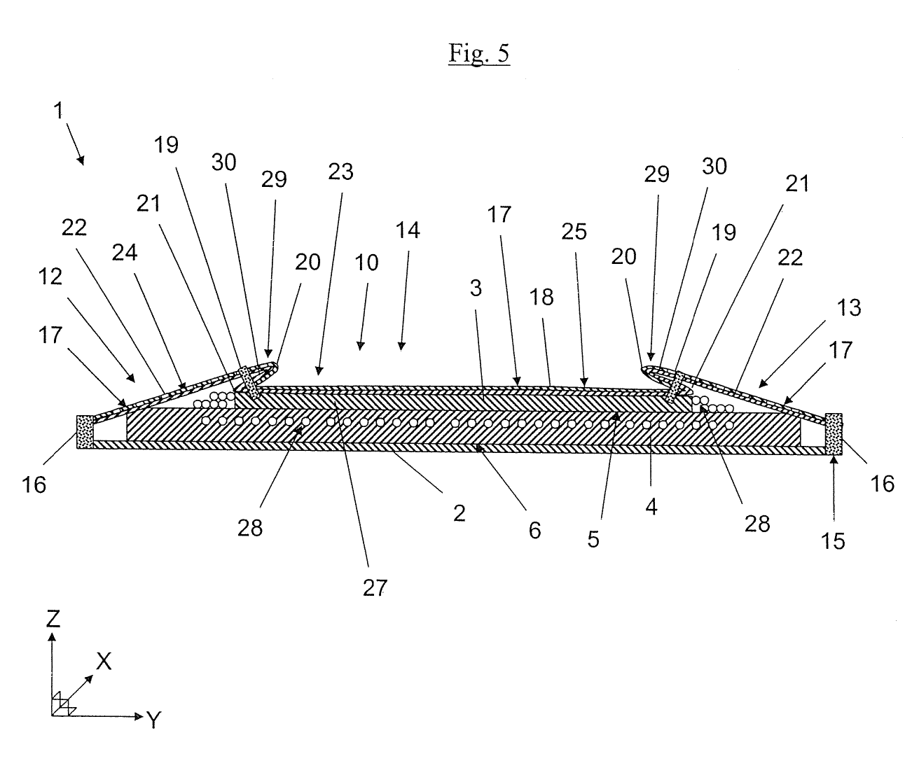

FIG. 5 depicts schematically a sectional view along the line A-A in FIG. 1 according to a fourth embodiment of the disclosure. FIG. 2 and FIG. 5 are identical apart from the position of the inner joining zones 19. FIG. 5 shows that the inner joining zones 19 are situated closer to the outer fold lines 21 than the inner fold lines 20. The position of the inner joining zone 19 adjacent to the outer fold line 21 provides an overlap of the top layer 3, which is locked in position, with the result that a large pleat 29 is formed in the top layer 3 between the inner joining zone 19 and the inner fold line. The pleat also forms a space 30 in the overlapping part that is present and faces towards the central zone. The pleat can be filled with a material to enable the pleat to function as a barrier, so-called “standing gathers”, which prevents liquid from being transported in a direction away from the central zone to the lateral zones. The material can consist of an absorption body which absorbs liquid that has passed through the top layer 3; or an elastic element which can form the top layer 3. Examples of elastic elements are elastic fibres, pieces of elastic foam, elastic layers or laminates, etc.

The disclosure is not restricted to the embodiments indicated above, but can be varied within the scope of the granted Patent Claims. It can be mentioned here by way of example that the absorbent product can lack a receiving layer, in which case the top layer does not need to be attached to the receiving layer via the inner joining zones, but can be attached at the inner joining zones directly to the absorption body. Another example is that the absorbent product constitutes a panty liner intended to absorb only small quantities of liquid, for example discharges, etc., and for this reason consists only of a top layer and a water-impermeable backing layer. The top layer in this case is attached only to the backing layer in the inner joining zones and/or in the outer joining zones.

A further example is that the top layer is attached to the edge part of the backing layer only via the outer joining zones, and not to the rest of the absorbent product. The inner joining zones thus comprise only the first layer of material and the second layer of material.

Accordingly, it is to be understood that the invention should not be considered as limited by the above description; rather the scope and limitations of the inventions are defined solely by the granted claims, and equivalents thereof.

1. An absorbent product comprising a top layer comprising a laminate of a first layer of material containing openings and a second layer of material, the absorbent product having two lines extending in the longitudinal direction, which divide the product in a lateral direction in such a way that the product has a first lateral zone, a second lateral zone and positioned between them a central zone,
wherein the first layer of material covers the two lateral zones and is arranged to face a user during use, and in that the second layer of material covers both the two lateral zones and the central zone, the laminate in the top layer being folded over itself along an inner fold line and an outer fold line, which outer and inner fold lines are present in a boundary zone between one of the lateral zones and the central zone in such a way that an overlap is formed with a Z-shaped cross section, the overlapping parts being joined together adjacent to at least one of the lines extending in the longitudinal direction in an inner joining zone.

2. The absorbent product according to claim 1, wherein the overlapping part of the Z-shaped fold is oriented inwards towards the central zone.

3. The absorbent product according to claim 1, wherein the top layer comprises a first part including the first layer of material containing a material with openings of a first size and a second part including the first layer of material containing a material with openings of a second size.

4. The absorbent product according to claim 3, wherein the first part of the top layer covers at least the two lateral zones, and in that the second part of the top layer covers the central zone, the top layer being folded in such a way that first part overlaps a section of the second part in such a way that the overlap forms the Z-shaped cross section.

5. The absorbent product according to claim 1, wherein the first layer of material covers only the lateral zones, or in that the first layer of material covers the lateral zones and the central zone, but that the first layer of material comprises the openings only in the lateral zones.

6. The absorbent product according to claim 1, wherein the first layer of material comprises a material in the lateral zones that is the same as in the central zone, or in that the material in the lateral zones differs from the material in the central zone.

7. The absorbent product according to claim 1, wherein the fold lines are essentially parallel and are situated at a distance from one another both in the lateral sense and in the vertical sense.

8. The absorbent product according to claim 1, wherein the inner joining zone is positioned between and at a distance from the outer and the inner fold lines.

9. The absorbent product according to claim 1, wherein the absorbent product comprises a receiving layer, to which the top layer is attached via the inner joining zone.

10. The absorbent product according to claim 1, wherein the absorbent product comprises a backing layer, to which the top layer is attached in an outer joining zone.

11. The absorbent product according to claim 10, wherein the absorbent product comprises an absorption body positioned between the backing layer and the top layer.

12. The absorbent product according to claim 1, wherein the second layer of material comprises holes which partially overlap openings in the form of holes in the first layer of material at least in the lateral zones.

13. The absorbent product according to claim 1, wherein the second layer of material lacks openings at least in the lateral zones.

14. The absorbent product according to claim 1, wherein the openings comprise holes and/or slits.

15. A method for the manufacture of an absorbent product, the absorbent product comprising a top layer comprising a laminate of a first layer of material containing openings and a second layer of material, the absorbent product having two lines extending in the longitudinal direction, which divide the product in a lateral direction in such a way that the product has a first lateral zone, a second lateral zone and positioned between them a central zone,
wherein the first layer of material covers the two lateral zones and is arranged to face a user during use, and in that the second layer of material covers both the two lateral zones and the central zone,
the method comprising:
folding the laminate of top layer over itself along an inner fold line and an outer fold line, which outer and inner fold lines are present in a boundary zone between one of the lateral zones and the central zone in such a way that an overlap is formed with a Z-shaped cross section, the overlapping parts being joined together adjacent to at least one of the lines extending in the longitudinal direction in an inner joining zone.

16. The method according to claim 15, wherein the folding is executed in such a way that the overlapping part of the Z-shaped fold assumes a direction in towards the central zone.

17. The method according to claims 15, wherein the top layer comprises a first part including the first layer of material containing a material with openings of a first size and a second part including the first layer of material containing a material with openings of a second size.

18. The method according to claim 17, wherein the first part covers at least the two lateral zones, and in that the second part covers the interjacent central zone, the top layer being folded in such a way that first part overlaps a section of the second part in such a way that the overlap forms the Z-shaped cross section.

19. The method according claim 15, wherein the first layer of material covers only the lateral zones, or in that the first layer of material covers the lateral zones and the central zone, but that the first layer of material comprises the openings only in the lateral zones.

20. The method according to claim 15, wherein the first layer of material comprises a material in the lateral zones that is the same as in the central zone, or in that the material in the lateral zones differs from the material in the central zone.

21. The method according to claim 15, wherein the folding is executed in such a way that the fold lines are essentially parallel and are situated at a distance from one another both in the lateral sense and in the vertical sense.

22. The method according to claim 15, wherein the inner joining zone is positioned between and at a distance from the outer and the inner fold lines.

23. The method according to claim 15, wherein the absorbent product comprises a receiving layer, to which the top layer is attached via the inner joining zone.

24. The method according to claim 15, wherein the absorbent product comprises a backing layer, to which the top layer is attached via an outer joining zone.

25. The method according to claim 24, wherein the absorbent product comprises an absorption body positioned between the backing layer and the top layer.

26. The method according to claim 15, wherein the second layer of material comprises openings which partially overlap the openings in the first layer of material at least in the lateral zones.

27. The method according to claim 15, wherein the second layer of material lacks openings at least in the lateral zones.

28. The method according to claim 15, wherein openings contain holes and/or slits.
